(19) United States
(12) Patent Application Publication
(10) Pub. No.: US 2025/0011703 A1
(43) Pub. Date: Jan. 9, 2025

(54) PROFILE AND MODULE FOR USE IN PHOTOBIOREACTOR

(71) Applicant: ALGIECEL APS, Copenhagen (DK)

(72) Inventors: Henrik BUSCH-LARSEN, Copenhagen (DK); Niels-Henrik NORSKER, Copenhagen (DK); Robert LARSEN, Copenhagen (DK); Christian KJØLBY, Copenhagen (DK)

(21) Appl. No.: 18/712,444

(22) PCT Filed: Nov. 22, 2022
(86) PCT No.: PCT/EP2022/082818

§ 371 Date: May 22, 2024

(30) Foreign Application Priority Data
Nov. 23, 2021 (DK) PA202101111
May 20, 2022 (DK) PA202200479

(51) Int. Cl. C12M 1/00; C12M 3/00

(52) U.S. Cl. CPC C12M 31/02; C12M 21/02; C12M 23/44; C12M 23/46

(57) ABSTRACT

The present invention relates to a profile comprising an elongated element extending in a longitudinal direction along a centre line and at least 3 planes extending in the longitudinal direction of the elongated element and surrounding the centre line of the elongated element, wherein at least one light source is placed on at least one of the 3 planes of the elongated element.

### TECHNICAL FIELD OF THE INVENTION

The present invention relates to a profile for improving light emission to a plurality of reactor tubes and at the same time controlling temperature, a module comprising and positioning a plurality of the profiles and a photobioreactor comprising one or more modules for culturing a phototrophic microorganism in a closed environment.

### BACKGROUND OF THE INVENTION

Algae are photogenic microorganisms and grow by consuming carbon dioxide (CO₂) using the mechanism described as photosynthesis.

Algae are a diverse group of photogenic microorganisms that play an important role in the biosphere and algae are characterized by a high growth rate potential compared to other typical energy crops. They are increasingly used in foods, nutraceuticals, agriculture, environmental protection, medicine and energy production.

Many systems have been described for production of algae and products derived from this production often have a low efficiency, low productivity and low yields due to suboptimal production process parameters, or the production facilities require large areas causing difficulties in up- or down-scaling of the production capacity.

Parameters of the production process, such as nutrients supplied during production, concentration of nutrients, pH, inorganic carbon supply including CO₂ and hydrogen carbonate and temperature may be important, high productivity of good-quality algae, optimal growth rate and high productivity of algae products may depend on factors like distribution and availability of light with the appropriate wavelength and intensity.

Algae are a potential source of renewable energy and a raw material for production of biofuels, cosmetics, and a significant source of food and feed. Unfortunately, the production method presently available are very energy-intensive making production of algae uneconomical, with low productivity and a large footprint and with difficulties in up- or down-scaling of the production capacity.

Thus, there is a need in the industry for improved systems with lower footprint and methods for growing photogenic microorganisms, like algae, to improve productivity and make the production of algae and products derived therefrom more economically interesting.

A photobioreactor according to the invention is used for growing photogenic microorganisms, like algae, and providing products e.g. for human consumption or animal or pet consumption which is possible due to the hygienic and controlled manner in which production takes place. A photobioreactor according to the invention may easily be adapted to optimize production for a specific microorganism or to optimize purity of a product by controlling light, flow, temperature and reducing footprint. The photobioreactor may be used for production of products for human consumption, products for animal or pet consumption, aqua culture feed, which product may comprise unsaturated fatty acids, in particular oil containing omega-3, omega-6 and/or omega-9 fatty acids, or proteins or pigments such as carotenoids, or biofuels.

### SUMMARY OF THE INVENTION

Thus, an object of the present invention relates to improving of a tubular photobioreactor to enable cost-efficient production of microalgae for the mentioned purposes. In particular, it is an object of the present invention to provide an adequately distributed and adequate amount of irradiation to the production tubes in a compact design, preferably, contained in a standard and high cube container that solves the above-mentioned problems of the prior art with cost-efficiency of algae production for the stated applications.

An aspect of the present invention relates to a profile (1) comprising an elongated element (2) extending in a longitudinal direction along a centre line C and at least 3 planes (13) extending in the longitudinal direction of the elongated element (2) and surrounding the centre line (C) of the elongated element (2), wherein at least one light source (4) is placed on at least one of the 3 planes (13) of the elongated element (2).

A further aspect of the present invention relates to a profile (1) comprising an elongated element (2) extending in a longitudinal direction along a centre line (C) and at least 4 planes (13) extending in the longitudinal direction of the elongated element (2) and surrounding the centre line (C) of the elongated element (2), wherein at least one light source (4) is placed on at least one of the 4 planes (13) of the elongated element (2) wherein the elongated element (2) comprises a hollow cavity (5) extending throughout the length of the elongated element (2) and wherein the hollow cavity (5) is adapted to hold a fluid, such as water or the like.

Another aspect of the invention relates to a profile (1) comprising an elongated element (2) extending in a longitudinal direction along a centre line C and comprising at least 3 blades (3) extending radially from the centre line (C) of the elongated element (2), wherein at least one light source (4) is placed on or adjacent to the surface of the elongated element (2) in one or in more or in each space between two blades (3).

A further aspect of the present invention relates to a module comprising at least one profiles (1) according to the present invention, which module further comprises at least one reactor tube (6) and fastening means (7) locking the reactor tube (6) in position relative to the at least one profiles (1).

An even further aspect of the present invention relates to a module comprising at least one profiles (1) comprising an elongated element (2) extending in a longitudinal direction along a centre line (C) and at least 4 planes (13) extending in the longitudinal direction of the elongated element (2) and surrounding the centre line (C) of the elongated element (2), wherein at least one light source (4) is placed on at least one of the 4 planes (13) of the elongated element (2), which module further comprises at least two reactor tubes (6) and fastening means (7) locking the reactor tube (6) in position relative to the at least one profiles (1).

Yet an aspect of the present invention relates to a frame (14) comprising one or more holes for locking, holding and/or stabilising one or more photobioreactor tube and one or more profile according to the present invention.

A further aspect of the present invention relates to a frame (14) comprising one or more holes for locking, holding and/or stabilising one or more photobioreactor tube and one or more profile comprising an elongated element (2) extending in a longitudinal direction along a centre line (C) and at least 3 planes (13) extending in the longitudinal direction of the elongated element (2) and surrounding the centre line (C) of the elongated element (2), wherein at least one light source (4) is placed on at least one of the 4 planes (13) of the elongated element (2).

An even further aspect pf the present invention relates to a photobioreactor comprising at least one module according to the present invention.

Yet an aspect of the present invention relates to a container comprising a module and/or a photobioreactor according to the present invention.

Another aspect of the present invention relates to the use of the photobioreactor according to the present invention as being part of an installation producing carbonaceous material, solely or partially derived from carbon dioxide and/or hydrogen carbonate.

### BRIEF DESCRIPTION OF THE FIGURES

FIG. 1 shows a cross-sectional view of a first embodiment of a profile having 4 blades according to the invention.

FIG. 2 shows a second embodiment of a profile having 6 blades according to the invention.

FIG. 3 shows a first embodiment of a part of a module according to the invention comprising a profile having 4 blades according to the present invention.

FIG. 4 shows an embodiment of the present invention wherein the blades are very short and not used for guiding the reactor tubes.

FIG. 5 shows an embodiment of a frame according to the present invention suitable for holding, locking and stabilising photobioreactor tubes and profiles according to the present invention

FIG. 6 show second embodiment of a part of a module according to the present invention comprising photobioreactor tubes and profiles according to the present invention held and stabilised by a frame according to the present invention.

FIG. 7 shows a combination of modules according to the invention.

FIG. 8 shows a further combination of modules according to the invention.

FIG. 9 illustrates a production plant according to which a module combination may operate.

The present invention will now be described in more detail in the following.

### DETAILED DESCRIPTION OF THE INVENTION

Thus, an embodiment of the present invention relates to a profile comprising an elongated element extending in a longitudinal direction along a centre line C and at least 3 planes (preferably 4 planes) extending in the longitudinal direction of the elongated element and surrounding the centre line of the elongated element, wherein at least one light source is placed on at least one of the 3 planes (preferably 4 planes) of the elongated element.

In an embodiment of the present invention the elongated element may comprise a hollow cavity extending throughout the length of the elongated element. Preferably, the hollow cavity may be adapted to hold a fluid, such as water or the like.

Preferably, the centre line (C) may be a straight line.

According to any embodiment of the present invention, the elongated element may comprise a hollow cavity extending throughout the length of the elongated element.

Preferably, the hollow cavity constitutes in the range of 20-95% of the cross-sectional area of the elongated element, such as in the range of 25-90%, e.g. in the range of 30-85%. The hollow cavity may have a constant cross-sectional area where the profile of the cross-sectional area e.g. is round, or the hollow cavity may have a cross-sectional area which varies in the longitudinal direction of the elongated element.

According to any embodiment of the present invention, the hollow cavity may have an inlet at a first end of the elongated element and an outlet at the opposite second end of the elongated element.

According to any embodiment of the present invention, the hollow cavity may be adapted to hold a fluid. Preferably, the fluid may be water, cooling fluid (such as glycol), or the like, i.e. the hollow cavity may be closed or fluid tight-when sealed in both ends.

The hollow cavity may preferably be adapted to hold and transport a fluid, such as water or the like, from one end of the elongated element to the other end of the elongate element.

According to any embodiment of the present invention, multiple light sources may be placed with a predetermined distance along either the width of the space between two blades and/or the length of the elongated element.

According to any embodiment of the present invention, the predetermined distance between the multiple light sources in the length of the elongated element may be in the range of 1-800 mm, such as in the range of 5-600 mm, e.g. in the range of 10-500 mm, such as in the range 20-400 mm, e.g. in the range of 30-300 mm, such as in the range of 40-200 mm, e.g. in the range of 50-125 mm, such as in the range of 60-90 mm, e.g. in the range of 70-80 mm, such as about 75 mm.

According to any embodiment of the present invention, the hollow cavity may be positioned centrally inside the elongated element, and/or the hollow cavity comprises baffles or the like increasing turbulence inside the cavity when fluid flows through the cavity from inlet to outlet. Fluid flowing inside the hollow cavity may be used for temperature control i.e. either heating or cooling of material or units positioned along the elongated element, e.g. of the planes or in the space between the blades, normally the fluid will be used to cool the elongated element as light sources on the elongated elements release heat, which should be removed to prolong lifetime and electric efficiency of the light sources and/or to reduce negative effects on the growth in the reaction tubes. However, in a very cold environment or in a situation where a high temperature is needed, a fluid flowing in the hollow cavity may be used to heat elongated elements.

According to any embodiment of the present invention, the elongated element and/or the blades may be made of a metal such as aluminium or a metal containing polymer.

A second aspect of the present invention relates to a module comprising at least two profiles according to the first aspect, which module further comprises at least one reactor tube and fastening means locking the reactor tube in position relative to the at least two profiles.

In the present context the term “reactor tube(s)” and “photobioreactor tube(s)” may be used interchangeably.

According to any embodiment of the present invention, the fastening means may either be part of the profiles e.g. part of the blades of the profiles, and/or the fastening means may be constituted by a frame surrounding the complete module.

In an embodiment of the present invention the fastening means may be provided as part of the blades, as an extension to the blades, or as a frame.

According to any embodiment of the present invention, each photobioreactor tube May have an inlet for fluid at a first end and an outlet for fluid at an opposite second end.

According to any embodiment of the present invention, the photobioreactor tube(s) may be made of a translucent material such as glass or a polymer such as an acrylate polymer also known as acrylics or polyacrylates.

A third aspect of the present invention relates to a photobioreactor comprising at least one module according to the second aspect, being part of an installation producing carbonaceous material, solely or partially derived from carbon dioxide and/or hydrogen carbonate. Preferably, when the installation producing carbonaceous material, is partially derived from carbon dioxide and/or hydrogen carbonate the carbonaceous material, may be predominantly derived from carbon dioxide and/or hydrogen carbonate.

According to any embodiment of the third aspect, the photobioreactor may be part of an installation comprising a degassing device downstream of the photobioreactor and an inlet for CO₂ and an inlet for nutrients upstream of the photoreactor.

According to any embodiment of the third aspect, the temperature inside the photobioreactor may be controlled e.g. by heat exchanging the photobioreactor with a fluid, such as a liquid e.g. water, or a gas. Preferably, the fluid may be flowing in the cavity of the profile according to the present invention.

The hollow cavity may not be in fluid contact with the at least one light source.

The number of light sources per distance may be determined based on the by the size, the effect and/or the efficiency of the light source. The number of light sources, e.g. LED's or OLED's with varying effects and efficiencies may be adjusted accordingly to provide the desired effect to the photobioreactor.

In general, the light source may comprise or be constituted of a multiplicity of diodes, in one direction there may be between 1 to 4 cm distance per diode, i.e. there may be 25 to 100 diodes per meter elongated element 2, e.g. between 40 to 100 diodes per meter elongated element 2, and preferably between 50 to 80 diodes per meter elongated element.

An embodiment of the present invention a profile comprising six planes having diodes emitting light in six directions, may have 6 directions×(25 to 100 diodes)=between 150 to 600 diodes per meter elongated element.

To increase the light transfer from light source to the reactor tubes, the surface(s) of a blade facing towards a space with a light source may be coated with a glossy or mirror like material reflecting the incoming light.

The first embodiment of a profile also comprises a hollow cavity positioned and extending along the centre line C inside the elongated element. Normally such a hollow cavity extends in the full length of the elongated element.

In general, the purpose of the hollow cavity is to instigate improved temperature control in the light sources and in a reactor comprising profiles. The hollow cavity is therefore normally adapted to hold a fluid, i.e. the hollow cavity comprises fluid tight walls and is provided with at least one inlet for fluid and at least one outlet for fluid. E.g., an inlet is position at a first end of the elongated element and an outlet is positioned at the opposite second end of the elongated element.

The hollow cavity is shown as having a round cross-section, but the hollow cavity may have any cross-section as long as the fluid flowing inside the hollow cavity is isolated from the surroundings at the outer surface of the elongated element. To increase heat transfer between the fluid inside the hollow cavity and the surroundings, it might be advantageous to provide the walls of the hollow cavity with a pattern or baffles to increase heat transfer to the flow inside the hollow cavity.

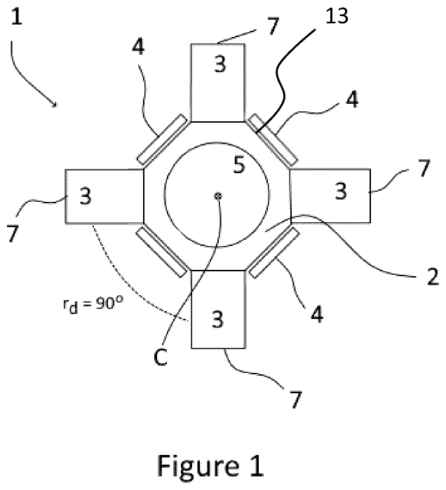

The hollow cavity of the embodiment shown in FIG. 1 might be between 7 and 9 mm in diameter.

An inlet of each reactor tube of a module may be connected to a manifold which manifold distributes feed equally to the inlets of all reactor tubes of a module. Also, an outlet of each reactor tube of a module may be connected to a manifold thereby collecting product from all the reactor tubes of a module.

In an embodiment of the present invention, the elongated element comprises at least 3 planes (preferably creating a triangular cross-sectional structure of the elongated element) extending in the longitudinal direction of the elongated element and surrounding the centre line of the elongated element, such as at least 4 planes (preferably creating a square cross sectional structure of the elongated element), e.g. at least 5 planes (preferably creating a pentagonal cross sectional structure of the elongated element), such as at least 6 planes (preferably creating a hexagonal cross sectional structure of the elongated element). Preferably, the elongated element comprises 6 planes extending in the longitudinal direction of the elongated element and surrounding the centre line of the elongated element, creating a hexagonal cross-sectional structure of the elongated element.

The profile according to the present invention may comprise 3 planes or more, such as 4 planes or more, e.g. 5 planes or more, such as 6 planes or more. Preferably the profile according to the present invention comprises 6 planes. The planes of the profile may be evenly distributed around the elongated element creating an equal number of edges (e.g. 6 planes results in 6 edges), one edge between each 2 and 2 planes. In an embodiment no edges are provided with a blade. In another embodiment of the present invention one or more (but not all) edges may be provided with a blade, such as 2 or more, e.g. 3 or more, such as 4 or more, e.g. 5 or more, such as 6 edges or more.

Preferably, the elongated element comprises a hollow cavity extending throughout the length of the elongated element.

In an embodiment of the present invention the elongated element may comprise a hollow cavity and wherein the hollow cavity is located in, or substantially in, the centre of the elongated element.

The hollow cavity may preferably be surrounded by the at least 3 planes of the elongated element. Thus, the at least 3 planes may form an enclosed cavity of the elongated element with openings in the ends of the elongated element.

The elongated element may comprise several hollow cavities (preferably separate cavities) extending throughout the length of the elongated element.

The plane according to the present invention may relate to a specified portion of the surface of the elongated element, preferably where a light source may be attached to the profile.

The surface may be the outer face of the elongated element.

In an embodiment of the present invention a light source may be placed on at least one of the planes of the elongated element.

Preferably, multiple light sources may be placed on at least one of the planes of the elongated element. In an embodiment of the present invention multiple light sources may be placed with a predetermined distance along the length of the elongated element on at least one of the at least 3 planes.

In an embodiment of the present invention, the multiple light sources may be placed with a predetermined distance along the length of the elongated element on each of the at least 3 planes, such as on each of the at least 4 planes, e.g. on each of the at least 5 planes, such as on each of the at least 6 planes.

The at least 3 planes may be flat planes or curved planes. Preferably, the curved planes may be a convex curved structure.

When present, the blades may have a length in the range of 1 mm to 25 cm, determined from the surface of the elongated element to the tip of the blade.

In one embodiment of the present invention the blades may have a length below 5 cm, such as a length below 4 cm, e.g. a length below 3 cm, such as a length below 2 cm, e.g. a length below 1 cm, such as a length below 0.5 cm, e.g. a length below 0.2 cm.

In another embodiment of the present invention the blades may have a length above 5 cm, such as in the range of 5-25 cm, e.g. in the range of 7-23 cm, e.g. in the range of 9-20 cm. In the event the blades are in the range of 5-25 cm, the blades may be used for guiding at least one reactor tube in the space between two blades.

A preferred embodiment of the present invention relates to a module comprising at least one profile according to the present invention, which module further comprises at least one reactor tube and fastening means locking the reactor tube in position relative to the at least one profile.

Preferably, at least one profile in the module according to the present invention may be directly illuminating at least 3 reactor tubes, preferably at least 4 reactor tubes, more preferably at least 5 reactor tubes, even more preferably 6 reactor tubes.

In an embodiment of the present invention the module may comprise the combination of a frame and a profile having at least 3 blades. Preferably, the blades in this module may have a length below 5 cm as defined above.

Preferably, at one reactor tube in the module may be directly illuminated by at least 2 (preferably 3) light sources on at least 2 (preferably 3) different profiles.

In an embodiment of the present invention wherein at least one profile in the module may be directly illuminating at least 3 reactor tubes, preferably at least 4 reactor tubes, more preferably at least 5 reactor tubes, even more preferably 6 reactor tubes, and wherein at one reactor tube in the module may be directly illuminated by at least 2 (preferably 3) light sources on at least 2 (preferably 3) different profiles.

The module according to the present invention may comprise at least 3 reactor tubes placed adjacent to each other and distributed by an angle of about 120°. Preferably, the module according to the present invention may comprise multiple reactor tubes collected in groups of at least 3 reactor tubes placed adjacent to each other and distributed by an angle of about 120°. A reactor tube may be shared between two groups of at least 3 reactor tubes, more preferably a reactor tube may be shared between 3 groups of at least 3 reactor tubes.

The module according to the present invention may comprise at least 4 reactor tubes placed adjacent to each other and distributed by an angle of about 120° and around a central reactor tube. Preferably, the module according to the present invention may comprise multiple reactor tubes collected in groups of 4 reactor tubes placed adjacent to each other and distributed by an angle of about 120° and around a central reactor tube. A reactor tube may be shared between two or more groups of at least 4 reactor tubes, more preferably a reactor tube may be shared between 3 groups of at least 3 reactor tubes more preferably a reactor tube may be shared between 4 groups of at least 4 reactor tubes.

In an embodiment of the present invention the module comprises more reactor tubes than profiles. Preferably the module comprises 10% more reactor tubes than profiles, such as more than 20%, e.g. more than 30%, such as more than 40%, e.g. more than 50%, such as more than 60%, e.g. more than 70%, such as more than 80%, e.g. more than 90%, such as more than 100%.

The number of planes comprising a light source in the module may be the same as the number of reactor tubes, preferably number of planes comprising a light source may be higher than the number of reactor tubes, may be at least 1.5 times the number of reactor tubes, such as at least 2 times the number of reactor tubes, e.g. at least 3 times the number of reactor tubes, such as at least 3.5 times the number of reactor tubes, e.g. at least 4 times the number of reactor tubes.

In an embodiment of the present invention the fastening means may be a frame.

A preferred embodiment of the present invention relates to a frame comprising one or more holes for locking, holding and/or stabilising one or more photobioreactor tube and one or more profile.

Preferably, the frame may be provided as a plate provided with holes dimensioned for receiving one or more profiles according to the present invention and/or holes dimensioned for receiving one or more reactor tubes.

In an embodiment of the present invention the module may comprise two or more frames, such as 3 or more frames, e.g. 4 or more frames, such as 5 or more frames, e.g. 6 or more frames, such as 7 or more frames, e.g. 8 or more frames, such as 9 or more frames, e.g. 9 or more frames.

The frames provided in the module may be substantially evenly distributed along the length of the reactor tube and/or the module. It may be preferred that for each 10-200 cm, such as each 20-150 cm, e.g. each 30-100 cm, such as each 40-60 cm, e.g. about each 50 cm reactor tube or module a frame may be placed.

The frames may be provided to hold and/or stabilize the reactor tube for movements and to provide an organised and/or optimized distribution of the reactor tubes and the profiles of the module. Using the profile according to the present invention may also allow easy repairment and/or exchange of the individual parts of the module, e.g. by individual removal or individual inspection of tubes or profiles.

The frames may be provided as a single frame or as multiple frames provided in layers which may be stacked and locked together. When multiple frames are stacked and locked together each frame may hold 1 layer of tubes or more, such as 2 layers of tubes or more, e.g. 3 layers of tubes or more, such as 4 layers of tubes or more, e.g. 5 layers of tubes or more, such as 7 layers of tubes or more, e.g. 10 layers of tubes or more, such as 10 layers of tubes or more, e.g. 20 layers of tubes or more, such as in the range of 1-20 layers of tubes, e.g. in the range of 2-15 layers of tubes, such as in the range of 3-10 layers of tubes, e.g. in the range of 4-5 layers of tubes.

The number of frames may depend on the length of the reactor tubes and/or the profile.

A preferred embodiment of the present invention relates to a frame comprising one or more holes for locking, holding and/or stabilising one or more photobioreactor tube and one or more profile according to the present invention.

Preferably, the one or more holes comprises one or more photobioreactor holes for introducing one or more photobioreactor tube, preferably, one photobioreactor hole may be provided for each of the photobioreactor tubes present.

Preferably, the one or more holes comprises one or more profile holes for introducing one or more profile, preferably, one profile hole may be provided for each of the profiles present.

In an embodiment of the present invention the one or more holes comprises one or more photobioreactor holes and one or more profile holes.

In a further embodiment of the present invention the photobioreactor holes comprises a diameter in the range of 20-80 mm, such as in the range of 30-60 mm, e.g. in the range of 35-50 mm, such as about 40 mm and/or wherein the profile holes comprises a diameter in the range of 2-25 mm, such as in the range of 5-20 mm, e.g. in the range of 7-15 mm, such as about 10 mm.

Preferably, the photobioreactor holes may be circular photobioreactor holes. Preferably, the photobioreactor holes comprise a diameter in the range of 20-80 mm, such as in the range of 30-60 mm, e.g. in the range of 35-50 mm, such as about 40 mm.

Preferably, the profile holes may be circular photobioreactor holes or hexagonal holes. Preferably, the profile holes comprise a diameter in the range of 2-25 mm, such as in the range of 5-20 mm, e.g. in the range of 7-15 mm, such as about 10 mm.

The frame may have a thickness in the range of 0.1-5 cm, such as in the range of 0.3-3 cm, e.g. in the range of 0.5-1 cm.

The frame according to the present invention may comprise at least 3 photobioreactor holes placed adjacent to each other and distributed by an angle of about 120° relative to each other.

The frame according to the present invention may comprise at least 4 photobioreactor holes placed adjacent to each other and distributed by an angle of about 120° relative to each other and around a central photobioreactor hole.

In an embodiment of the present invention the frame comprises more photobioreactor holes than profile holes. Preferably the frame comprises 10% more photobioreactor holes than profile holes, such as more than 20%, e.g. more than 30%, such as more than 40%, e.g. more than 50%, such as more than 60%, e.g. more than 70%, such as more than 80%, e.g. more than 90%, such as more than 100%.

Another embodiment of the invention relates a profile comprising an elongated element extending in a longitudinal direction along a centre line and comprising at least 3 blades extending radially from the centre line of the elongated element, wherein at least one light source is placed on or adjacent to the surface of the elongated element in one or in more or in each space between two blades. Normally the centre line is a straight line.

According to any embodiment of the present invention, the blades may extend from the surface of the elongated element, and the blades may extend in a straight direction from the elongated element.

According to any embodiment of the present invention, the blades may extend from the surface of the elongated element along the entire length of the elongated element along a straight line in the longitudinal direction of the elongated element, alternatively, the blades may extend from the elongated element along a part of the length of the elongated element along a straight line in the longitudinal direction of the elongated element. By providing a profile having blades extending radially from the elongated element for only part of the length of the elongated element—i.e. a blade is not a continuous piece of material but may appear as parts protruding radially from the surface of the elongated element—it is possible to limit the amount of material used for construction of the profile.

According to any embodiment of the present invention, the profile may comprise more than two blades, or more than 3 blades, or the profile may comprise less than 10 blades, or less than 7 blades, or the profile may comprise 3, 4, 5 or 6 blades extending radially from the surface of the elongated element.

According to any embodiment of the present invention, the distance between two neighbouring blades at the surface of the elongated element may be the same for all neighbouring blades.

According to any embodiment of the present invention, one photobioreactor tube may be placed between two blades of at least one profile, e.g. one photobioreactor tube may be placed between two blades of two or more profiles. When a photobioreactor tube is placed between two blades (3) of two or more profiles, then the photobioreactor tube may receive light from (be illuminated by) two or more light sources which decreases the need for creating turbulence inside the photobioreactor tube.

In general, each profile may hold a number of reactor tubes. According to an embodiment of the present invention the number of reactor tubes held by one profile equals the number of blades of the profile.

In general, a number of, or each blade, or elongated element of a profile may comprise fastening means securing, locking or stabilising a position of one profile relative to one, two or more profiles.

In general, the radial distance between two neighbouring blades and the diameter or outer cross-section of a reactor tube placed between the two neighbouring blades, may be adapted to position the reactor tube being between the two neighbouring blades at an optimum distance from a light source positioned on the surface of the elongated element. An optimum distance is the distance where light enter the reactor tube through the transparent wall and covers the largest possible volume with highest possible intensity. An optimum distance is the distance where light enter the reactor tube through the transparent wall and covers the largest possible volume with highest possible intensity.

The intensity decreases with distance, but the spreading of the light from the light source increases with distance and therefore the volume of mass inside the reactor tube being subjected to light increases.

The optimum distance between light source and outer wall surface of reactor tube may normally be between 5-30 mm, such as between 6 and 25 mm, e.g. between 7 and 20 mm, such as between 8-15, e.g. between 9-13 mm, e.g. about 11 mm.

A preferred embodiment of the present invention relates to a photobioreactor comprising at least one module according to the present invention.

The module may comprises at least one profile which may be illuminating at least 2 reactor tubes, preferably at least 3 reactor tubes, more preferably at least 4 reactor tubes, even more preferably at least 5 reactor tubes, most preferably at least 6 reactor tubes.

In an embodiment of the present invention the module does not comprise at least one profile submerged into the reactor tube.

A photobioreactor (PBR) is a bioreactor utilizing a light source to cultivate phototrophic microorganisms. These organisms use photosynthesis to generate biomass from light and carbon dioxide and include plants, mosses, macroalgae, microalgae, cyanobacteria and purple bacteria. A photobioreactor system according to the invention works as a closed system having controlled inlets of gas and fluid media, also microorganism or algae strains present inside the reactor is controlled.

The photobioreactor according to the present invention may comprise at least one module according to the present invention, such as at least two modules, e.g. at least 3 modules, such as at least 4 modules, e.g. at least 5 modules, such as at least 6 modules, e.g. at least 7 modules, such as at least 8 modules, e.g. at least 9 modules.

Modules may be combined, preferably, provided in fluid connection, to large reactor units e.g. reactor units being around 10 meters long and adapted to fill out the inside space of a container.

In an embodiment of the present invention the container may be a 20 feet container, a 40 feet container or a 45 feet container, preferably a 40 feet container.

In an embodiment of the present invention the container may be a high cube container.

It may be possible to easily scale up the production by adding one or more container comprising combined modules and the containers combined may be made in fluid contact allowing fermentation media to flow directly from the modules or the photobioreactor in one container to the modules or a photobioreactor in another container and soon. One or more product outlets may be introduced along the flow path provided by the one or more fluid connected modules, photobioreactors, and/or containers.

The outlets may be coupled to a downstream processing equipment/process line, which may be provided in a container too.

Use of the photobioreactor according to the present invention as being part of an installation producing carbonaceous material, solely or partially derived from carbon dioxide and/or hydrogen carbonate.

In the following the invention will be further described based together with references to the drawings.

FIG. 1 shows a cross-sectional view of a first embodiment of a profile (1) according to the invention. A central line (C) extend perpendicular to the cross-sectional view in the complete length of an elongated element (2) and therefore appears as a point in FIG. 1. The central line (C) is a straight line.

The profile (1) may also comprises a hollow cavity (5) positioned and extending along the center line (C) inside the elongated element (2). Normally such a hollow cavity (5) extend in the full length of the elongated element (2).

In general, the purpose of the hollow cavity (5) may be to instigate improved temperature control of the elongated element (2), in the light sources (4), and/or in a reactor comprising profiles (1). The hollow cavity (5) may therefore normally be adapted to hold a fluid, i.e. the hollow cavity (5) comprises fluid tight walls and is provided with at least one inlet for fluid and at least one outlet for fluid. E.g., an inlet is positioned at a first end of the elongated element (2) and an outlet is positioned at the opposite second end of the elongated element (2).

The hollow cavity (5) is shown as having a round cross-section, but the hollow cavity (5) may have any cross-section as long as the fluid flowing inside the hollow cavity (5) may be isolated from the surroundings at the outer surface of the elongated element (2). To increase heat transfer between the fluid inside the hollow cavity (5) and the surroundings, it might be advantageous to provide the walls of the hollow cavity (5) with a pattern or baffles to increase the heat transfer to the fluid inside the hollow cavity (5).

The hollow cavity (5) of the embodiment shown in FIG. 1 might be between 7 and 11 mm in diameter.

The shown embodiment in FIG. 1 comprises 4 blades (3) extending in the longitudinal direction of the elongated element (2) in a direction parallel to the central line (C). Each blade (3) also extends in a direction radial to the central line (C). The radial angle (ra) between two neighbouring blades (3) of this embodiment is constant for all neighbouring blades (3), and the constant value of (ra) is 90°. In general, the blades (3) may be constituted of a lightweight material e.g. a lightweight metal with heat conduction capacity such as aluminium. Preferably, the blades (3) and the elongated element (2) may be made from the same material. In order to save weight, each blade (3) may be constituted of a series of parts or protrusions which series of parts or protrusions have a shorter length in direction of the central line (C) than the complete length of the elongated element (2), while all parts or protrusions constituting a blade (3) extends in the same radial direction.

A light source (4) is placed in each of the planes (13) at the space between two neighbouring blades (3), in general, there might not be light sources in all planes (13) and in all spaces between blades (3), but normally placing light sources (4) in all planes, and spaces, may increase flexibility when using the profile (1) for construction of a larger module and increase productivity in a final photobioreactor. In general, the light source (4) might be of any type but preferable of a type having a high power to light efficiency such as LED.

The dimension of the blades (3) i.e. the length in the radial direction and the width in a direction perpendicular to the radial direction, may determine the distance between the light source (4) and also the dimension of the blades (3) is adapted to hold an elongated reactor tube with a circular cross-section such as a photobioreactor tube 6.

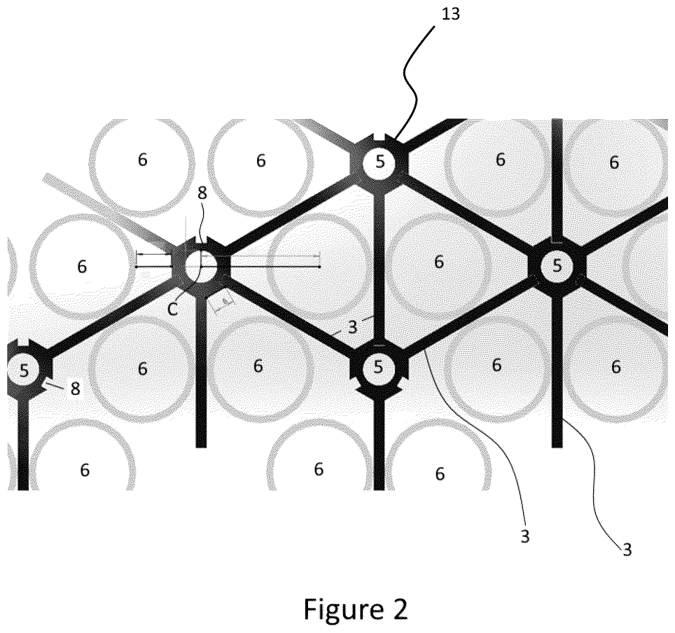

The length of a blade (3) in the radial direction may correspond to the radius of an elongated reactor tube with a circular cross section (6). For the embodiment of FIG. 2, the distance from one centre line (C) to a neighbouring centre line may be 2×the radius of the elongated reactor tube. I.e., if the width of a blade is 3 mm and the radius of an elongated reactor tube is 15 mm, then the cross-sectional maximum dimension of a profile (1) having 4 blades mounted with constant radial distance (embodiment of FIG. 1) may be 2×15+3 mm=33 mm.

Each light source (4) may be positioned on or adjacent to the plane (13) of the elongated element (2), in the shown embodiment the outer contact of the elongated element (2) is shown as a plane surface, however, the surface may have a different cross-section, it may e.g. be concave or convex or be provided with grooves which may be an advantage if the profile (1) is cooled or heated by an air flow passing around the light source (4). In a preferred embodiment, the outer surface of the elongated element (2) between two neighbouring blades (3) are adapted to fit the inner surface of a light source (4) e.g. be a straight plane in order to obtain close contact and optimum heat transfer between the material of the central part of the elongated element (2) and the light source (4).

FIG. 2 shows a cross-sectional view of a second embodiment of a profile (1) according to the invention. FIG. 2 shows an embodiment of section of a module where five elongated elements (2) have been combined by common blades (3), i.e., one blade (3) is attached to two elongated elements (2) at an attachment position (8) on each elongated element (2). According to this embodiment, each elongated element (2) comprises six fastening position for blades (3) and fastening means at each fastening position. The fastening positions may be longitudinally extending grooves where a blade (3) may be mounted by pushing the blade (3) into position either from an open end of the groove or by pushing the blade (3) radially towards the centre of the elongated element (2) into the groove. The (r_{d}) between two neighbouring blades (3) is 60° and the space around each elongated element (2) is split into 6 rooms, where each room can accommodate an elongated tube (6).

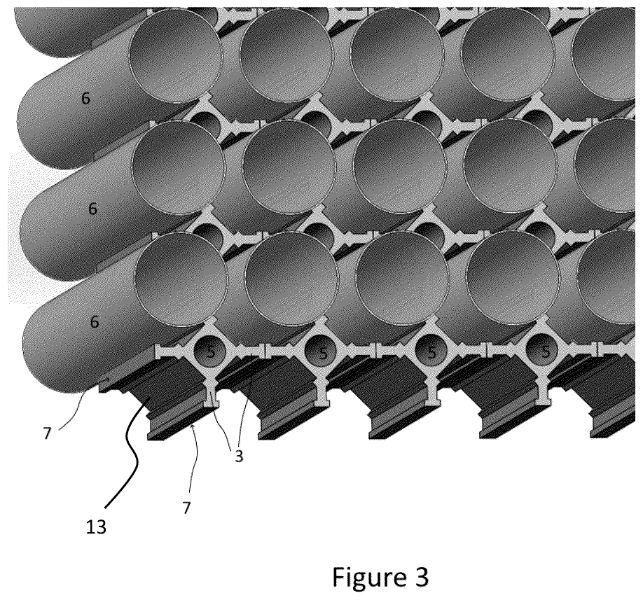

FIG. 3 shows a view of a combination of profiles (1) each having four blades (3) and reactor tubes (6) forming a section of a module (17). The end of each blade (3) functions as fastening means (7) and is either in contact with a neighbouring profile (1) or a not shown frame keeping the profiles (1) in correct relative position. FIG. 3 illustrates a section of a module (17) which can be constructed of a number of profiles (1) and a number of reaction tubes (6). In principle, any number of reaction tubes (6) may be combined into a module (17) and a number of modules (17) may then be combined into a reactor unit such as a photobioreactor.

According to the embodiment of FIG. 3, the fastening means (7) may be as simple as a flat end surface of the blades (3) e.g. in combination with a frame surrounding all reactor tubes (6) and profiles (1) of a module. When a group of profiles (1) is placed side-by-side within a frame, the profiles (1) will be locked in the direction perpendicular to the longitudinal direction of the elongated elements (2) of the profiles (1).

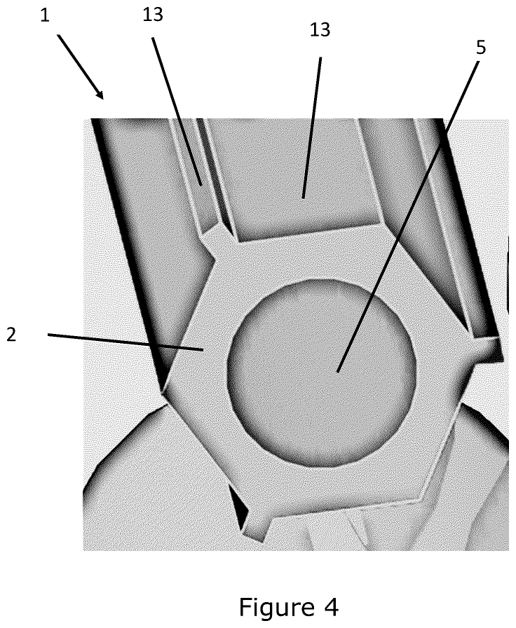

FIG. 4 shows a further embodiment of the profile 1 according to the present invention. The profiles (1) are shown with a combination of blades (3) and without blades and with 6 planes (13). A light source (not shown) may be attached to at least one of the planes (13), preferably, light sources are placed on all 6 planes (13) of the elongated element (2).

The profile (1) may also comprise a hollow cavity (5) positioned and extending along the center line inside the elongated element (2). Normally such a hollow cavity (5) extends in the full length of the elongated element (2).

In general, the purpose of the hollow cavity (5) may be to instigate improved temperature control of the elongated element (2), in the light sources (4), and/or in a reactor comprising profiles (1). The hollow cavity (5) may therefore normally be adapted to hold a fluid, i.e. the hollow cavity (5) comprises fluid tight walls and is provided with at least one inlet for fluid and at least one outlet for fluid. E.g., an inlet is position at a first end of the elongated element (2) and an outlet is positioned at the opposite second end of the elongated element (2).

The hollow cavity (5) is shown as having a round cross-section, but the hollow cavity (5) may have any cross-section as long as the fluid flowing inside the hollow cavity (5) may be isolated from the surroundings at the outer surface of the elongated element (2). To increase heat transfer between the fluid inside the hollow cavity (5) and the surroundings, it might be advantageous to provide the walls of the hollow cavity (5) with a pattern or baffles to increase turbulence of the flow inside the hollow cavity (5).

The hollow cavity (5) of the embodiment shown in FIG. 4 might be between 7 and 11 mm in diameter.

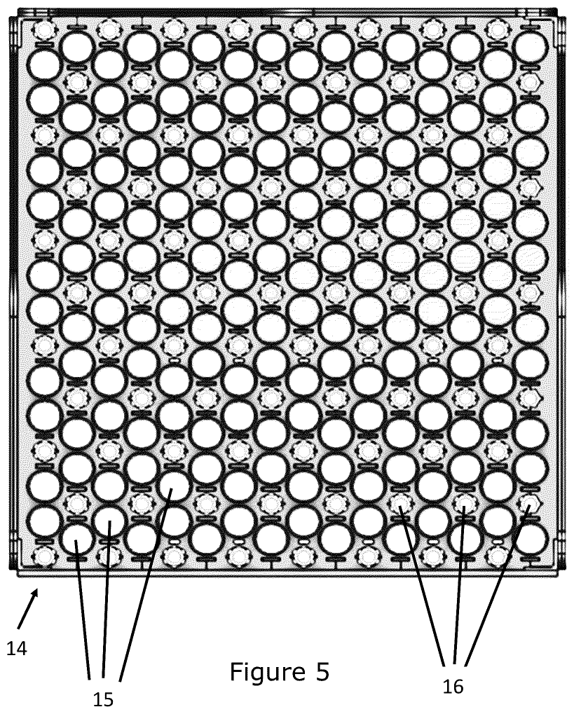

FIG. 5 shows a frame (14) for holding, locking and/or stabilising the reactor tubes and profiles. The frame (14) may comprise multiple photobioreactor holes (15) for introducing photobioreactor tubes and multiple profile holes (16) for introducing profiles according to the present invention.

One profile hole (16) in the frame (14) according to the present invention may be adjacent to 6 photobioreactor holes (15).

One photobioreactor holes (15) in the frame (14) may be adjacent to 3 different profile holes (16) and 3 photobioreactor holes (15).

The frame (14) comprises multiple groups of 3 photobioreactor holes (15) placed adjacent to each other and distributed by an angle of about 120°.

The frame (14) comprises multiple groups of 4 photobioreactor holes (15) placed adjacent to each other and distributed by an angle of about 120° and around a central reactor tube.

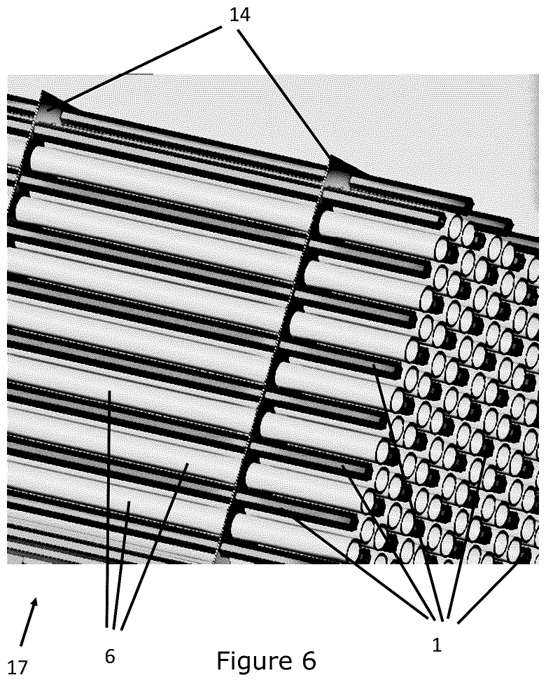

FIG. 6 shows a section of a module (17) comprising a number of elongated reactor tubes (6) and a number of profiles (1) held, locked and/or stabilised by two frames (14). The frame is provided with multiple photobioreactor holes for introducing multiple photobioreactor tubes (6) and multiple profile holes for introducing multiple profiles (1) according to the present invention.

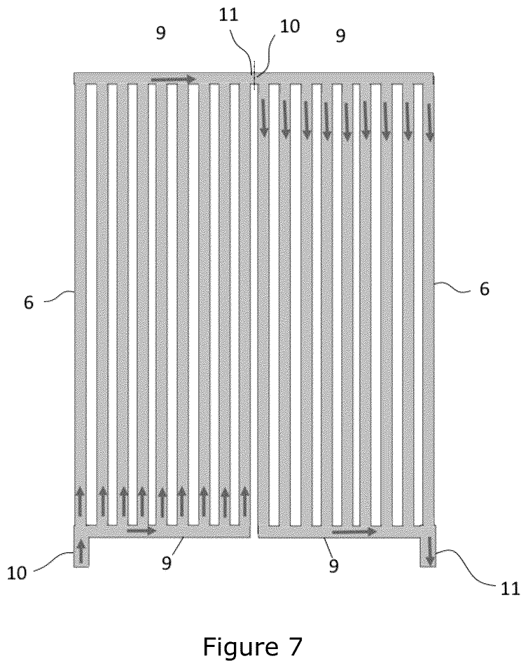

FIG. 7 shows an example of a module comprising one layer of reactor tubes 6, i.e. all reactor tubes 6 are positioned next to each other. The profiles 1 are not included in the figure but would be present between two neighbouring reactor tubes 6 in a working embodiment. The module comprises a first manifold inlet 10 from which the feed is distributed into the inlets of nine parallel reactor tubes 6. At the end of the nine reactor tubes 6 the product leaves the reactor tubes 6 through a reactor tube outlet and enters into a second manifold 9. The product from all nine parallel reactor tubes 6 are collected in the manifold 9 and leaves the manifold 9 through the manifold outlet 11 and entering a further manifold 9 via the manifold inlet 10. From this manifold 9 the product is distributed as feed to another nine parallel reactor tubes 6 and subjected to further reaction. Then the further reacted product is collected in a manifold and let to the outlet 11 from the manifold 9.

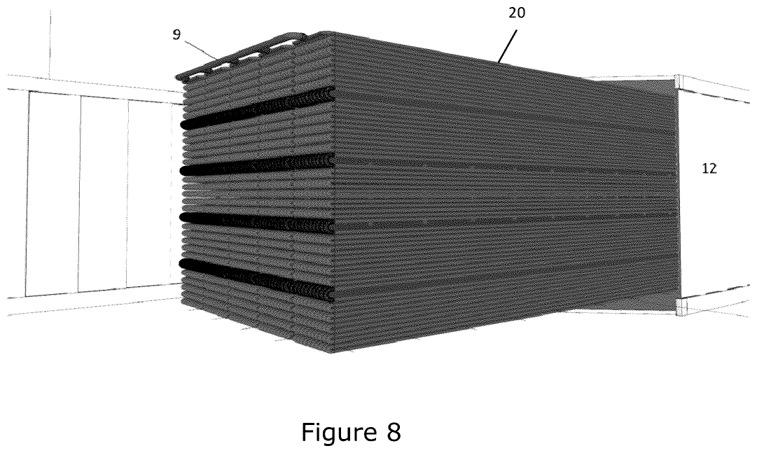

FIG. 8 shows a container (12) comprising a photobioreactor (20) comprising a manifold (9). The photobioreactor (20) may be provided from a number of modules. The number of modules may preferably be 1, 2, 4, 9, 16, or 25. The container shown in FIG. 8 comprises 25 modules. The container (12) may be a 20 feet container, a 40 feet container, or a high cube container, preferably a 40 feet container. Up-scaling may be easily done by adding one or more further containers to the container (12) which are fluidly connected allowing fermentation media to flow directly from a photobioreactor (20) in one container (12) to a photobioreactor (20) in another container (12).

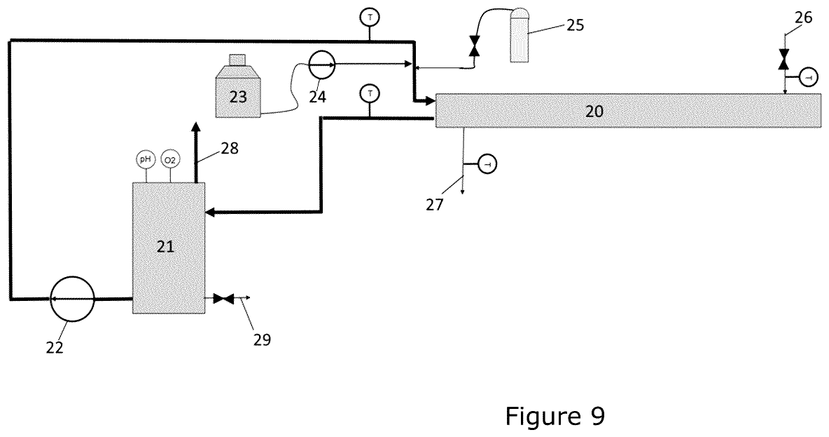

FIG. 9 shows a photobioreactor system where profiles and modules according to the present invention may be used.

The photobioreactor shown in FIG. 9 comprises a photobioreactor 20, a degassing tank 21, a circulation pump 22, a tank 23 with fresh nutrient fluid, a feed pump 24 pumping fresh nutrient into the circulating fluid, a CO₂ tank 25 from which CO₂ may be added to the circulating fluid.

During operation fluid media comprising one or more strains of phototrophic microorganisms flows through reactor tubes 6 inside the photobioreactor 20 and a light source 4, also placed inside the photobioreactor, emits light into the reactor tubes 6. A circulation pump 22 forces the fluid medial to circulate out of photobioreactor 20 and back into the photobioreactor 20, normally via manifold outlets and inlets.

The temperature of the fluid media passing out of the photobioreactor 20 and the temperature of the fluid media passing into the photobioreactor 20 may be measured and controlled. A heat exchange media, normally a liquid media such as water, is pumped into the photobioreactor 20 via an inlet 26 for heat exchange media and exits the photobioreactor 20 via an outlet 27 for heat exchange media. The temperature inside the photobioreactor may be controlled by controlling the inlet temperature of the heat exchange media.

From the photobioreactor 20 the fluid media is pumped to a degassing tank 21 where oxygen is removed from the media and pH is increased. The degassing tank 21 may have an outlet 29 for fluid near the bottom for taking samples of the content. Before the fluid media is pumped back to the photobioreactor 20, nutrient and carbon dioxide is added to the fluid media.

It should be noted that embodiments and features described in the context of one of the aspects of the present invention also apply to the other aspects of the invention.

| Ref. no. | Reference name |
|---|---|
| 1 | Profile |
| 2 | Elongated element |
| 3 | Blade |
| 4 | Light source |
| 5 | Hollow cavity |
| 6 | Elongated reactor tube, Elongated photobioreactor tube |
| 7 | Fastening means |
| 8 | Attachment position |
| 9 | Manifold |
| 10 | Manifold inlet |
| 11 | Manifold outlet |
| 12 | Container |
| 13 | Plane |
| 14 | Frame |
| 15 | Photobioreactor holes |
| 16 | Profile holes |
| 17 | Section of a module |
| 20 | Photobioreactor |
| 21 | Degassing tank |
| 22 | Circulation pump |
| 23 | Nutrient tank |
| 24 | Feed pump |
| 25 | CO2 tank |
| 26 | Inlet for cooling/heating media |
| 27 | Outlet for cooling/heating media |
| 28 | Outlet for O2 |
| 29 | Outlet for fluid |
| r_{d} | Radial distance |
| C | Central line extending in the longitudinal direction of an |
| | elongated element |

1. A profile (1) comprising an elongated element (2) extending in a longitudinal direction along a centre line (C) and at least 4 planes (13) extending in the longitudinal direction of the elongated element (2) and surrounding the centre line (C) of the elongated element (2), wherein at least one light source (4) is placed on at least one of the 4 planes (13) of the elongated element (2) wherein the elongated element (2) comprises a hollow cavity (5) extending throughout the length of the elongated element (2) and wherein the hollow cavity (5) is adapted to hold a fluid, such as water or the like.

2. The profile (1) according to claim 1, wherein the elongated element is provided with at least 5 planes extending in the longitudinal direction of the elongated element (2) and surrounding the centre line (C) of the elongated element (2).

3. The profile (1) according to claim 1, wherein the elongated element is provided with 6 planes extending in the longitudinal direction of the elongated element (2) and surrounding the centre line (C) of the elongated element (2).

4. A profile (1) according to claim 1, wherein multiple light sources (4) may be placed with a predetermined distance along the length of the elongated element (2) on at least one of the at least 4 planes.

5. The profile (1) according to claim 1, wherein the elongated element (2) comprises one hollow cavity (5) extending throughout the length of the elongated element (2).

6. A module comprising at least one profiles (1) comprising an elongated element (2) extending in a longitudinal direction along a centre line (C) and at least 4 planes (13) extending in the longitudinal direction of the elongated element (2) and surrounding the centre line (C) of the elongated element (2), wherein at least one light source (4) is placed on at least one of the 4 planes (13) of the elongated element (2), which module further comprises at least two reactor tubes (6) and fastening means (7) locking the reactor tube (6) in position relative to the at least one profiles (1).

7. The module, wherein the at least one profiles (1) is a profile according to claim 1.

8. The module according to claim 6, wherein the fastening means (7) is a frame (14).

9. A frame (14) comprising one or more holes for locking, holding and/or stabilising one or more photobioreactor tube and one or more profile comprising an elongated element (2) extending in a longitudinal direction along a centre line (C) and at least 3 planes (13) extending in the longitudinal direction of the elongated element (2) and surrounding the centre line (C) of the elongated element (2), wherein at least one light source (4) is placed on at least one of the 4 planes (13) of the elongated element (2).

10. The frame (14) according to claim 9, wherein the one or more holes comprises one or more photobioreactor holes (15) and one or more profile holes (16).

11. The frame (14) according to claim 10, wherein the photobioreactor holes (15) comprises a diameter in the range of 20-80 mm, such as in the range of 30-60 mm, e.g. in the range of 35-50 mm, such as about 40 mm and/or wherein the profile holes (16) comprises a diameter in the range of 2-25 mm, such as in the range of 5-20 mm, e.g. in the range of 7-15 mm, such as about 10 mm.

12. A photobioreactor comprising at least one module according to claim 6.

13. The photobioreactor according to claim 12, wherein module comprises at least one profile (1) which is illuminating at least 2 reactor tubes (6), preferably at least 3 reactor tubes (6), more preferably at least 4 reactor tubes (6), even more preferably at least 5 reactor tubes (6), most preferably at least 6 reactor tubes (6).

14. The photobioreactor according to claim 12, wherein the module does not comprise at least one profile submerged into the reactor tube.
